(19) United States
(12) Patent Application Publication
(10) Pub. No.: US 2009/0029879 A1
(43) Pub. Date: Jan. 29, 2009

(54) PROCESS FOR ENHANCED OIL RECOVERY USING A MICROBIAL CONSORTIUM

(75) Inventors: Bhupendra SONI, Sugar Land, TX (US); Banwari LAL, Rajasthan (IN); John Allen BABCOCK, Houston, TX (US)

Correspondence Address: CONLEY ROSE, P.C.;David A. Rose, P. O. BOX 3267, HOUSTON, TX, 77253-3267, US

(73) Assignee: GLORI OIL LIMITED, Houston, TX (US)

(21) Appl. No.: 12/166,705

(22) Filed: Jul. 2, 2008

Related U.S. Application Data
(60) Provisional application No. 60/949,402, filed on Jul. 12, 2007.

(51) Int. Cl. C09K 8/60; E21B 43/00; C12M 1/00; C12N 1/20

(52) U.S. Cl. 507/201; 166/66; 435/283.1; 435/253.6

(57) ABSTRACT

A process and related apparatus for enhancing the oil recovery from an oil well are described. An embodiment of the process generally includes selection of a candidate reservoir; collecting oil formation water samples under anaerobic conditions; selecting media and enriching the microbes derived from the formation water; characterizing and identifying the selected consortium; mass scale production of the selected consortium; anaerobically preparing a defined composition nutrient medium, transportation of the nutrient medium by ISO tankers and the consortium by a specially designed field laboratory unit to the selected well treatment site; injecting the medium and consortium into the reservoir of the well; closing the well for the proliferation of microbes for one to three weeks; and allowing the microbes to dislodge oil in the reservoir and thereby enhance recovery of oil from the well.

### CROSS-REFERENCE TO RELATED APPLICATIONS

This application claims the benefit under 35 U.S.C. § 119(e) of U.S. Provisional Patent Application No. 60/949,402 filed Jul. 12, 2007, the disclosure of which is hereby incorporated herein by reference.

### STATEMENT REGARDING FEDERALLY SPONSORED RESEARCH OR DEVELOPMENT

Not applicable.

### TECHNICAL FIELD

The invention generally relates to recovering petroleum crude oil from oil wells, and more particularly relates to compositions and processes that use downhole application of microbes to enhance recovery of oil, and to apparatus and compositions for carrying out such processes.

### BACKGROUND

Conventional oil production technologies are usually able to recover only about one-third of the total amount of oil in a subterranean reservoir. Ways to recover this residual oil to reverse the decline in domestic oil production and to increase the available oil reserves are of great interest today. Some conventional oil well treatments combine pressure with a change in viscosity of the oil and/or water that is present in the well. A diluent, CO₂ or steam is added to the reservoir to reduce the viscosity of the oil, thereby allowing it to be freed. Alternatively, viscosity-increasing additives such as polymers are added to the injection water so that the oil is preferentially dislodged. However, the application of CO₂ can be disadvantageous due to corrosion, and the use of steam is usually only effective in shallow reservoirs of low temperature. Chemical additives usually tend to be too costly to be commercially practical.

Another approach is to alter the surface tension and capillary forces so that part of the residual oil adhered to rock grains is released and produced. This may be achieved by alkaline flooding or by means of surfactants. These kinds of oil well treatments also tend to be commercially unattractive due to cost.

Still another type of enhanced oil recovery technique endeavors to dislodge oil from a formation through in situ combustion. This treatment entails pumping air or oxygen into the formation and igniting the gas/oil that is present. In theory, the heat produced will mobilize the lighter fractions as a combustion front moves steadily through the formation, with the heavier tars burning. In practice, however, it is almost impossible to control the progress since the gases tend to rise while the water present sinks, resulting in an uneven combustion front.

A different type of technology that has been investigated uses microorganisms such as bacteria to dislodge oil from a formation. This technology is called Microbially Enhanced Oil Recovery or “MEOR”. A MEOR process is potentially an economically attractive alternative to other processes for enhancing oil recovery because microbial processes do not consume large amounts of energy, as do thermal recovery processes, and microbial processes do not depend on the price of crude oil, in contrast to many chemical recovery processes. Because microbial growth occurs at exponential rates, it is theoretically possible to produce large amounts of useful metabolites quickly from inexpensive and renewable microbial resources.

A variety of MEOR methods have been described, including U.S. Pat. No. 3,332,487 (Jones), which describes an aerobic bacteria for enhancement in oil recovery. The process involves oxidation of oil to lower compounds. The partial oxidation products of these are low molecular weight alcohols, aldehydes, ketones and acids which act as viscosity reducing solvents for the remaining oil. Many of these are mutual solvents for oil and water, thus the method is said to permit a form of miscible fluid drive when water is introduced through the formation following the bacteria.

U.S. Pat. No. 4,522,261 (McInerney et al.) describes a method in which a pure culture of Bacillus licheniformis strain JF-2 (ATCC No. 39307) and a process for using said culture and the surfactant lichenysin produced thereby for the enhancement of oil recovery from subterranean formations. Lichenysin is an effective surfactant over a wide range of temperatures, pH's, salt and calcium concentrations.

U.S. Pat. No. 4,475,590 (Brown) describes a method of treating a subterranean oil-bearing reservoir having an in-situ microbial population therein. It is believed that the microorganisms originally enter the oil-bearing formations either in water injected in the formation during secondary waterfloods, or by invasion of water into the formation from adjacent aquifers after the reservoir has been partially depleted by oil production. The microorganisms subsist but do not proliferate to any appreciable extent in the reservoir because of nutritional deficiencies, primarily nitrogen and phosphorus.

U.S. Pat. No. 4,905,761 (Bryant) describes a microbial enhanced oil recovery process in which a combination of microorganisms is empirically formulated based on survivability under reservoir conditions and oil recovery efficiency, such that injection of the microbial combination may be made, in the presence of essentially only nutrient solution, directly into an injection well of an oil bearing reservoir having oil present at waterflood residual oil saturation concentration. The microbial combination is said to be capable of displacing residual oil from reservoir rock, which oil may be recovered by water flooding without causing plugging of the reservoir rock. The microorganisms are also said to be capable of being transported through the pores of the reservoir rock between said injection well and associated production wells, during water flooding, which results in a larger area of the reservoir being covered by the oil-mobilizing microorganisms.

U.S. Pat. No. 5,163,510 (Sunde et al.) describes a method of microbial enhanced oil recovery for recovering oil from an oil-bearing rock formation. A population of aerobic bacteria is introduced into the formation at a position spaced from a production borehole. The microorganisms are adapted to use oil as a carbon source. Pressurized injection water is introduced into the formation via an injection borehole, the water including a source of oxygen and mineral nutrients. The bacteria multiply using the oil as their main carbon source and the oxygen in the injection water as their main oxygen source. In so doing, they dissociate the oil from the rock formation and the dissociated oil is removed via the production borehole by the injection water.

U.S. Pat. No. 5,297,625 (Premuzic) describes the preparation of new, modified organisms, through challenge growth processes. The organisms are said to be viable in the extreme temperature, pressure and pH conditions and salt concentrations of an oil reservoir and suitable for use in microbial enhanced oil recovery. The modified microorganisms are used to enhance oil recovery and remove sulfur compounds and metals from the crude oil.

U.S. Pat. No. 5,363,913 (Jenneman et al.) describes the use of sequestering agents in addition to nutrients in a method for promoting microbial activity in subterranean formations at depth from the wellbore.

U.S. Pat. No. 6,758,270 (Sunde et al.) uses formation oil as the sole carbon source and nitrate as electron acceptor. The method comprising: injecting water containing a source of vitamins, phosphates and an electron acceptor into the formation, allowing micro-organisms in the form of anaerobic or facultative anaerobic bacteria, which are either already present in the formation or which are introduced simultaneously with the vitamins, phosphates and electron acceptor to multiply using the oil as their main carbon source thereby establishing a biomass layer which acts to dissociate the oil from the rock formation; and removing the dissociated oil via the outlet.

PCT International Patent Application Publication No. WO 05/005773 (Lal et al.) describes a certain microbial consortium containing three hyperthermophilic, barophilic, acidogenic, anaerobic bacterial strains for enhanced oil recovery from oil reservoirs where temperatures range from 70° C. to 90° C. and salt concentrations do not exceed about 5%. The microbial consortium produces a variety of metabolic products mainly CO₂, methane, biosurfactant, volatile fatty acids and alcohols in the presence of specially designed nutrient medium. These metabolic products increase sweep efficiency of crude oil from oil bearing pores of rock formation. A process for enhancing the oil recovery includes in situ application of that microbial consortium.

MEOR methods take advantage of the ability of microbes to produce products such as gases, surfactants, acids, solvents, and polymers/biomass for improving oil recovery. These products, in turn, can favorably alter oil and/or rock properties and thereby facilitate additional oil recovery from a well. To be successful, however, the selected microbes must be able to live and proliferate to the necessary level in the harsh reservoir environment. A drawback of many existing MEOR methods is that the microorganisms employed typically cannot survive at a temperature beyond 70° C. There is continued interest in developing ways to enhance oil recovery from oil wells using microbial treatment, particularly those which can be employed cost effectively.

### SUMMARY

From screening studies in which the best-performing microbe consortium are selected, it is also determined which mineral rich media formulations and which nutrient rich media formulations best support the growth of those microbes and also promote production of the desired metabolites. For instance, some metabolites may be effective for dislodging oil in the formation. In some instances, the particular combination of media components may serve to suppress growth of some of the indigenous microbes in the well while enhancing proliferation of the selected microbes. By appropriately screening and selecting the microbes and media components, a customized bulk medium formulation is developed for use with a selected microbial consortium in an oil reservoir that meets certain selection criteria. The selected consortium is grown in mass scale production. The defined medium for field application is prepared anaerobically using a specially designed mixing system and transportation of the nutrient medium is done using ISO tankers and the consortium by a specially designed field laboratory.

In some embodiments, a specific sequence is followed for injection of nutrient medium and the selected microbial consortium (inoculum) into the selected well to ensure that no contaminants are introduced and that the medium and inoculum remain anaerobic during the entire Process for Enhanced Oil Recovery by Microbial Consortium (“PEORMC”).

In accordance with certain embodiments of the invention, a process or method is provided for enhancing recovery of oil from a subterranean reservoir. The method comprises (a) selecting an oil well reservoir having a treatment zone and meeting the following criteria:

| | |
|---|---|
| Type of formation: | sandstone or carbonate |
| | with dense natural fracturing |
| Presence of gas cap: | no |
| Stage of exploitation: | mature |
| Depth of occurrence: | <10,000 ft |
| Reservoir temperature: | <90° C. |
| Reservoir pressure: | <300 Kg/cm² |
| Formation water pH: | 4-9 |
| Salinity of formation water: | <12% total salinity or <10% NaCl |

(b) providing an anaerobic inoculum comprising a selected consortium of anaerobic microbes obtained or derived from a reservoir that meets the criteria of (a), wherein the selected microbes have a predetermined growth rate at the reservoir temperature, have a predetermined minimum mineral and nutrient requirements for growth, and produce predetermined amounts and/or kinds of metabolites; (c) providing a volume of anaerobic nutrient medium formulated to support the growth of the consortium and meeting at least the predetermined minimum mineral and nutrient requirements, the volume being sufficient to fill the treatment zone of the oil well reservoir; (d) anaerobically combining the inoculum with the anaerobic nutrient medium to form an anaerobic bioslurry; (e) displacing the bioslurry into the reservoir treatment zone; and (f) shutting in the oil well and allowing the consortium to incubate in the zone, to enhance oil recovery from the well. In some embodiments, this comprises shutting in the oil well for about one to about three weeks, for example.

In step (d) of some embodiments, anaerobically combining the inoculum with the medium comprises anaerobically combining the inoculum with a portion of the anaerobic nutrient medium to form the anaerobic bioslurry while simultaneously pumping the medium into the well. For example, in some embodiments this includes injecting the consortium into the stream through a venturi valve configured to exclude air from the medium.

In step (b) of some embodiments, the microbes are initially obtained by anaerobically sampling the formation water of the reservoir by anaerobically introducing the sample directly from the reservoir tubing into a sterile container under cover of an inert gas. For example, in some embodiments, this includes aseptically injecting a reducing agent into the container under cover of the inert gas.

In some embodiments, the consortium comprises one or both of Clostridium bifermentans and Garciella petrolaria. In some embodiments, the pH of the formation water is in the range of about 6-8. In some embodiments, the formation water contains up to 12% (w/v) salt.

In step (a) of some embodiments, the reservoir additionally meets at least one of the following criteria:

| | |
|---|---|
| Reservoir rock permeability: | >25 md |
| Crude oil gravity: | >20°API |
| Reservoir oil viscosity: | <20 cp |
| Reservoir rock porosity: | >20%, in the case of sandstone |
| | formation only |
| Water cut: | 30-95% |
| Residual oil saturation: | >25% |
| Source of water: | Natural aquifer or water injection |
| Net pay thickness: | 10-12 meters or less |
| Producing zone: | single |
| Type of lift: | rod pump |
| Swelling clays in formation rock: | none |

In accordance with certain embodiments of the invention, a system is provided for anaerobically treating an oil reservoir. The system comprises: a transportable field laboratory assembly including: (1) a pumping unit comprising a triplex pump having a low pressure suction line configured for coupling to a feed line and a high pressure discharge line configured for coupling to an oil well inlet, (2) a laboratory unit comprising at least one anaerobic bioreactor having an outlet adapted for coupling to the discharge line, (3) optionally, a power unit containing a generator for powering the system; and (4) a housing enclosing at least the laboratory unit and the pumping unit.

In some embodiments, the system further comprises a plurality of instrument probes operationally positioned in the suction and discharge lines for measuring predetermined operational parameters during operation of the system; and a plurality of instruments in electronic communication with the probes, respectively, for receiving data from the probes and reporting information about the respective parameters.

In some embodiments, the instruments are configured for measuring and reporting at least one parameter selected from the group consisting of oxidation redox potential (ORP), temperature, pH, conductivity, pressure, and flow rate.

In accordance with certain other embodiments of the invention, a system for producing an anaerobic nutrient medium is provided which comprises: (a) a water processing unit comprising at least one water holding tank, and configured for providing a sterile water stream; (b) a chemical unit comprising: (1) a bulk chemical tank configured for receiving bulk chemicals and the sterile water stream, (2) at least one chemical holding tank configured for receiving a bulk chemical solution from the bulk chemical tank and for providing a chemical stream, and (3) a nutrient concentrate holding tank configured for receiving a blended stream and for providing a nutrient concentrate stream. The system further includes (c) a blending unit comprising a secondary chemical tank, a mixing device and a blending tank configured for receiving the chemical stream and the water stream, and for providing the blended stream; (d) an export unit comprising a dosing tank and a first manifold adapted for receiving the sterile water stream, the nutrient concentrate stream, and the blended stream, and for receiving a dosing stream from the dosing tank, the export unit also adapted for discharging a nutrient medium; (e) a second manifold in fluid communication with the water processing unit, the chemical unit, the blending unit, and the first manifold, and configured for receiving the sterile water stream; (f) a third manifold in fluid communication with the chemical unit and the blending unit, and configured for receiving a chemical solution from the chemical unit; and (g) a gas distribution assembly in fluid communication with the water tanks, chemical holding tanks, bulk chemical tank, nutrient concentrate tank, secondary chemicals tank, blending tank and dosing tank.

In some embodiments, the at least one chemical holding tank is in fluid communication with the third manifold. In some embodiments, the secondary chemical tank is configured for receiving the water stream and the blending tank is configured for receiving a secondary chemical stream from the secondary chemical tank. In some embodiments, the export unit further comprises a digital flow totalizer and a filtration membrane in fluid communication with the first manifold.

In accordance with certain embodiments of the invention, a system for producing an anaerobic nutrient medium is provided which comprises means for providing anaerobic sterile water; means for providing a bulk chemical solution; means for providing a secondary chemical solution; means for blending the water, bulk chemical solution, and secondary chemical solution, to provide a nutrient concentrate; means for transferring the water, bulk chemical solution, and secondary chemical solution into the blending means; means for holding the nutrient concentrate; means for exporting the nutrient concentrate; means for adjusting the concentration of the nutrient concentrate during exporting of the concentrate; and means for maintaining anaerobic conditions in the system.

In accordance with certain embodiments of the invention, a method of producing a nutrient medium is provided which comprises (a) providing an above-described system for producing an anaerobic nutrient medium; (b) in the chemical unit of the system, anaerobically preparing a bulk chemical solution comprising NaCl, MgCl₂, KH₂PO₄, KNO₃, NH₄Cl, and corn steep liquor; (c) in the blending unit of the system, anaerobically preparing a second chemical solution comprising casein peptone, malt extract, sodium acetate, a redox indicator and a reducing agent; (d) in the blending unit, anaerobically combining predetermined amounts of the first and second chemical solutions and water, to produce a nutrient concentrate; (e) anaerobically transferring the nutrient concentrate to the nutrient concentrate holding tank of the chemical unit; and (f) in the exporting unit, anaerobically exporting predetermined amounts of the sterile water and the nutrient concentrate into a receiving tank, to yield an anaerobic nutrient medium. In some embodiments, the method further includes (g) adjusting the pH and redox potential of the nutrient concentrate during exporting of the medium to the receiving tank. In some embodiments, the anaerobic nutrient medium in the receiving tank comprises the following ingredients and amounts: 5-100 g/L NaCl, 0.2-0.4 g/L MgCl₂; 0.2-0.4 g/L KH₂PO₄; 0.2-0.4 g/L KNO₃, 0.2-0.4 g/L NH₄Cl, 0.002-0.003 g/L Resazurin, 0.2-0.4 g/L Cysteine HCL, 0.5-1.5 g/L Sodium acetate, 1-10 g/L Corn steep liquor, 0.5-1.5 g/L Casein peptone, 0.5-1.5 g/L Malt extract, and water, up to a volume of about 40,000 to 150,000 liters.

In accordance with certain embodiments of the invention, an anaerobic sampling device is also provided which comprises (a) a sterile sample bottle including a pierceable septum; (b) a sample bottle container including a first injection needle coupled to a bleed line including a valve, and a second injection needle coupled to a sample inlet line, wherein the container is adapted for receiving the bottle and the first and second needles are adapted for piercing the septum; (c) a formation water line comprising a valve adapted for coupling to an oil well tubing flow line; (d) an inert gas inlet line including a valve adapted for coupling to a source of inert gas; and (e) a three-way valve operatively connected to the formation water line, the sample inlet line, and to a fluid bypass line. These and other embodiments and potential advantages of the invention will be apparent with reference to the following description and drawings.

### BRIEF DESCRIPTION OF THE DRAWINGS

FIG. 1 is a schematic box flow diagram of an improved process for enhancing oil recovery using a selected microbial consortia, in accordance with an embodiment of the invention.

FIG. 2 is a schematic diagram of a representative anaerobic sampling device in accordance with an embodiment of the invention.

FIG. 3 is a schematic process flow diagram of a blending system that is used in the preparation of media under anaerobic conditions in accordance with an embodiment of the invention.

FIG. 4 is a schematic diagram of a transportable field laboratory and pump assembly according to an embodiment of the invention.

FIG. 5 is a schematic process flow diagram of an oil well treatment in accordance with an embodiment of the invention.

### DETAILED DESCRIPTION

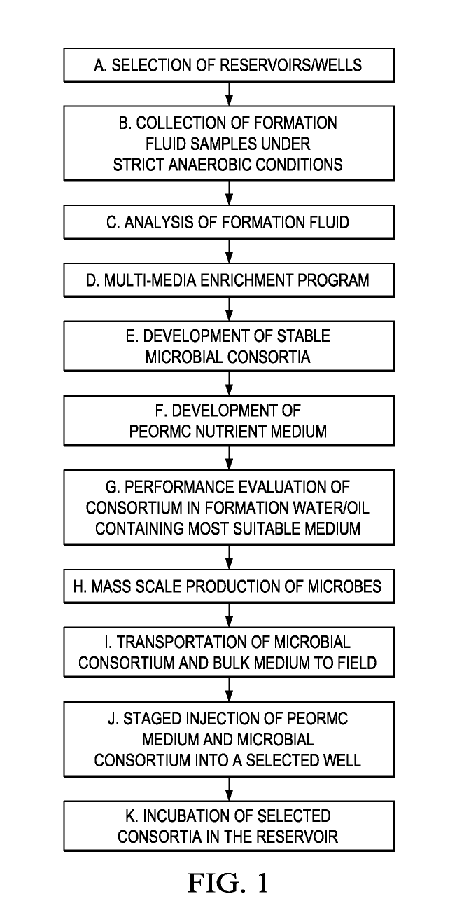

Referring to FIG. 1, an embodiment of a Process for Enhancing Oil Recovery by Microbial Consortium (PEORMC) generally includes (A) selection of one or more oil wells that are suitable candidates for application of the PEORMC by determining several broad rock and fluid parameters, and other well or reservoir characteristics of the well(s); (B) collection of one or more oil well formation fluid samples under strict anaerobic conditions; (C) analysis of the formation fluid samples; (D) carrying out a multi-media enrichment program; (E) developing from the formation water samples a stable consortium of anaerobic microorganisms selected to grow under conditions like those of the subterranean environment of the selected oil well(s). The microorganisms are selected based on such desirable characteristics as production of volatile fatty acids, solvents, biosurfactants, polymers, gases and biomass; (F) developing a special PEORMC nutrient medium to support growth of the selected microorganisms in the selected downhole environment; (G) evaluating the performance of a selected consortium in formation water/oil containing the most suitable medium from Step (F); (H) producing the selected microbial consortium in mass scale; (I) transporting the nutrient medium and the selected microbial consortium to the site of an oil well that is selected for treatment, while maintaining the medium and the consortium anaerobic; (J) mixing the nutrient medium with the selected microbial consortium to form a bioslurry, and then injecting the mixture into the oil well reservoir in specified stages, while maintaining both the medium and the consortium in an anaerobic environment; and (K) allowing the consortium to incubate in the formation to dislodge oil, to increase the flow of oil, and to enhance oil recovery from the treated well.

Selection of Reservoirs/Wells. In many embodiments, the broad formation and fluid properties of an oil well/reservoir that is suitable for application of PEORMC meet all of the criteria listed in Table 1:

TABLE 1
Primary Selection Criteria for Treatment Reservoirs/Wells

| Parameter | Criteria |
|---|---|
| Type of formation: | sandstone or carbonate with |
| | dense natural fracturing |
| Presence of gas cap: | no |
| Stage of exploitation: | mature |
| Depth of occurrence: | <10,000 ft |
| Reservoir temperature: | <90° C. |
| Reservoir pressure: | <300 Kg/cm² |
| Formation water pH: | 4-9, preferably 6-8 |
| Salinity of formation water: | <12% (total salinity) |
| | <10% (NaCl) |

In some applications, a suitable well may also have one or more additional properties which may increase the likelihood of success of the process for enhanced oil recovery using a microbial consortium. These may include, for example, reservoir rock permeability >25 md; crude oil gravity >20° API; reservoir oil viscosity <20 cp; reservoir rock porosity >20% (sandstone only); water cut 30-95%; residual oil saturation >25%; net pay thickness: 10-12 meters or less; producing from a single zone; type of lift: e.g., rod pump; no swelling clays in formation rock. Alternatively, if swelling clays are present in the rock, they may be inhibited during the PEORMC, using, for example, a product such as Treat-a-Clay™, manufactured by Envirochem, a Hidalgo Quelett Company, for example.

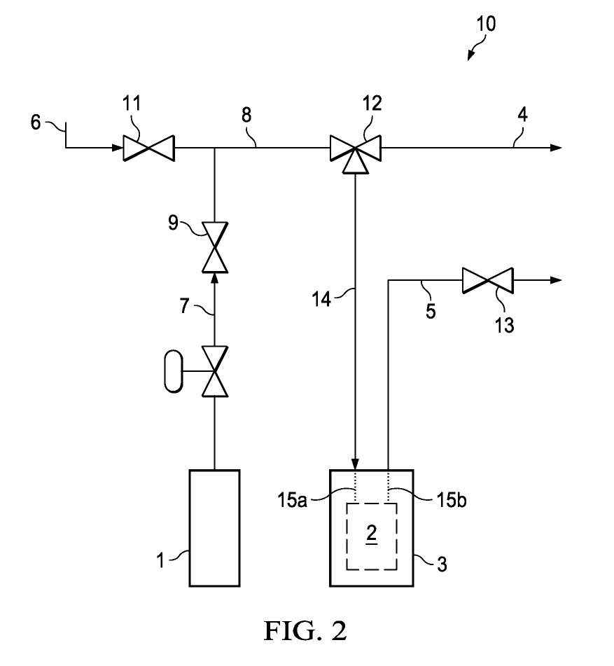

Collection of Water Samples Under Strict Anaerobic Conditions. The wells/reservoirs meeting the selection criteria are selected and become the candidates for application of the PEORMC. Water samples from the producing wells of these reservoirs are collected under strict anaerobic conditions. These samples become the source of isolation and identification of one or more microbes that are useful for PEORMC. In some embodiments, anaerobic sampling is accomplished using a specially designed sampling device equipped with a nitrogen flushing unit and equipped for addition of reducing agents into the sample collection vessels to further ensure maintenance of anaerobic conditions of the sample(s). For instance, one or more sterile containers are filled with the formation water while flushing the container with nitrogen. The reducing agents are aseptically added to the containers. The sealed containers are then taken to a laboratory for testing and processing. FIG. 2 is a schematic diagram of a representative sampling device, which may be constructed from commercially available components, for obtaining the anaerobic formation water sample. The sampling device 10 includes a sample bottle 2 with septum (not shown), a sample container 3 with injection needles (indicated by dotted lines 15a, 15b), a by-pass line 4, bleed line 5, formation fluid inlet 6, water line 8, nitrogen line 7 with valve 9. Line 7 is configured for attaching to the regulator valve of a nitrogen bottle 1. Lines 4, 8 and 14 connect to three-way valve 12. Line 7 includes a valve 9, line 8 includes a valve 11 and line 5 includes a valve 13, for controlling the flow of incoming nitrogen, formation fluid, and the nitrogen bleed, respectively.

Each well completion may have a unique configuration depending on the completion method, artificial lift equipment and design volume. For example the tubing and flow line configurations may be different for gas lift, beam lift, hydraulic lift and submersible pump completions. Tubing and flow line pipe diameter may differ from one well to another, depending on the volume of fluid that is being produced from a given oil well. Sampling device 10 is suitable for coupling to the oil flow line 6 of any type of well completion. When attaching the sampling device to a selected well, line 6 is always connected to the tubing flow line. In most embodiments, it is connected through an existing bleeder valve that is normally used to connect a pressure measuring gauge to the tubing flow line. For some applications, the user may have available a variety of pipe fittings so as to be able to connect a sampling device 10 to the oil flow line of any type of well completion configuration that is presented at the well field location.

The sampling process is generally carried out as follows: In the field, any necessary pipe fitting is first installed to adapt from the well tubing flow line to inlet 6 of device 10. Then, (A) the sampling device is connected in the field location by connecting the nitrogen bottle to line 7 and connecting the formation water inlet 6 to the tubing flow line of the well to be sampled; (B) the nitrogen regulator on the nitrogen bottle 1 is opened and flow valve 9 is opened; (C) the three-way valve 12 is adjusted to direct the flow of nitrogen from line 8 into line 14. Nitrogen flows through lines 7 and 8, is diverted through the three-way valve, and displaces nitrogen into sample bottle 2. Using the bleed line 5 and valve 13, nitrogen is allowed to flow out the by-pass line 5, thereby purging the container and water lines with nitrogen; (D) nitrogen flow is shut off, the three-way valve is closed, and the wellhead flow line is opened. Valve 11 is opened, allowing formation fluid to enter inlet 6, and flow through fluid line 8 and out by-pass line 4 until flow becomes representative of the formation fluid. In most situations, valve 11 is opened slowly in order to choke the flow of well fluids and prevent excessive flow rates from entering the sampling device; (E) the flow is then diverted through the three-way valve and displaces nitrogen in the sample bottle 2 to fill the bottle with well fluid, allowing the displaced nitrogen to exit via bleed line 5 and valve 13; and (F) the well fluid is then shut in and the sampling assembly is disconnected from the tubing flow line. Strict anaerobic conditions are thus maintained throughout the sampling operation. The sample bottle containing the formation fluid may be removed from the sample bottle container 3 for processing.

Analysis of Formation Water. In many instances, the formation fluid sample is allowed to stand several hours or overnight in the laboratory, to allow water/oil phase separation. The water phase is then sampled for analysis of at least the content of anions, cations, heavy metals, and organic acids. The sample is also tested for conductivity, specific gravity and total salinity using techniques and equipment as are known in the art. A portion of this water phase is also used for the enrichment process.

Analysis of Formation Oil In some applications, the oil phase that separates from the formation water in the foregoing step is analyzed for viscosity using standard techniques and equipment that are known in the art. Additional oil characterization may be conducted, including some or all of the following:
1. Sulfur % and API Gravity, Ni/V Ratio
2. Stable carbon isotopes of the saturates and aromatic fractions (δ₁ aromatics, δ₁ saturates)
3. Bulk composition (Saturates, aromatics, NSO compounds, Resinoids and Asphaltenes) using nC₅ and nC₆ as solvent
4. Whole Oil Gas Chromatography (C₄ to C₄₀)
   4.1 Isoprenoids (pristane/nC₁₇, phytane/nC₁₈)
5 C₆-C₇ Gas Chromatography
   5.1 Thompsons alteration vectors (Toluene/nHeptane vs n-Heptane/Methycyclohexane)
   5.2 Ring preference analysis (as described in Mango, F. D., et al, 1994, GCA 58:895 for example)
   5.3 P1 (nC₇), P2 (mono branched C₇ Alkanes) and P3 (poly branched C₇ alkanes) analysis
   5.4 P3 vs Primesum analysis—Shell Ratios type analysis
6. Gas Chromatography—Mass Spectrometry on Aromatic Hydrocarbons fraction
   6.1 One, two and three ring components (MN, DMN, TMN, P, MP, DMP)
   6.2 Aromatic biomarkers
7. High Temperature Gas Chromatography of Whole Oil (C₄ to C₇₆)
   7.1 High molecular-weight waxy fractions
8. Gas Chromatography—Mass Spectrometry of the Saturate Hydrocarbon Fraction
   8.1 Saturate biomarker analysis m/s 191, m/z 217, m/z 171, m/z 177, m/z 218, m/z 259, m/z 205, m/z 178, m/z 192, m/z 206, m/z 231, m/z 253
   8.2 Terpenoids/Triterpane-Steranes-Hopanes/hopanoids fingerprints (m/z 191)
   8.3 C₂₉ 25-norhopane and C₂₆-C₃₄ 25-norhopanes (m/z 171)
   8.4 Sum MNT/C23 Tricyclic terpanes ratio, MNT: Methyl Naphthalenes

The aptitude of oils as a feasible substrate for a PEORMC program using compositional parameters can be addressed by three main groups of indicators: The first one deals with the determination of natural levels of biodegradation suffered by target oils during geological time frames. A second set of parameters relates to the actual partition of different hydrocarbon families residing in the oil sample as potential carbon substrate serving for microbial metabolism associated with PEORMC processes. Finally, there are a number of indicators related to the multiple-parametric correlation between compositional changes occurring by man-made microbial processes and the improvement in physical properties with impact in oil productivity in a particular reservoir-extractive environment. This framework of analyses permits the fine comparison between Pre-PEORMC and Post-PEORMC changes for relevant molecular entities, compositions and biomarkers during design and monitoring stages.

The proportion of saturated hydrocarbons in sample oils is by far high enough to serve as the sole carbon substrate during a PEORMC process based on bio-cracking mechanisms. The level of bio-alteration operated on N-alkanes needs to be correlated with rheological measurements to infer improvements in differential oil productivity based on untreated and inoculated samples. The relevance of n-alkanes relies on the fact that these types of hydrocarbon are the first metabolized by selected microorganisms with biocracking functionality, rapidly increasing lighter components by simultaneous reduction operated on heavier ends of the molecules. Such compositional shift from high alkanes to low alkanes is also reflected by a corresponding large impact on the apparent viscosities of the treated oils, and on the modifications to the original rheology model for the treated oils.

Multi-Media Enrichment Schemes. Screening and selection of the microorganisms are performed using at least four different formulations of media, including various specially formulated nutrient rich media, nutrient rich media with minerals, and nutrient media with different carbon sources. For instance, a set of screening media may include (1) nutrient rich media, (2) nutrient rich media with minerals, (3) nutrient media with glucose, and (4) nutrient media with sucrose. Each of the media contains a concentration of sodium chloride that approximates the salt level in the oil well to be treated. In some media formulations, oil is used as the sole carbon source. The ingredients that are combined to make an exemplary set of screening media, and their amounts, are listed in Tables 2-6.

TABLE 2
Nutrient-Rich Medium

| Constituents | Amount (g/L) |
|---|---|
| Tryptone | 5-20 |
| Yeast extract | 3-8 |
| Malt extract | 5-10 |
| Sodium lactate | 2-10 |
| Sodium Acetate | 2-10 |
| Sodium chloride | 10-100 |
| Cysteine Chloride | 0.05-0.15 |
| pH | 7-7.5 |

TABLE 3
Medium Containing Minerals and Nutrients

| Ingredients | Amount (g/L) |
|---|---|
| Sucrose | 5-15 |
| Yeast extract | 1-3 |
| Corn steep liquor | 2-12 |
| Sodium acetate | 0.1-1.5 |
| NaCl | 5-150 |
| MgCl₂ | 0.1-0.3 |
| KH₂PO₄ | 0.1-0.3 |
| KNO₃ | 0.1-0.3 |
| NH₄Cl | 0.1-0.3 |
| Cysteine hydrochloride | 0.05-0.15 |

TABLE 4
Medium Containing Glucose as Carbon
Source and Containing Nutrients

| Ingredients | Amount (g/L) |
|---|---|
| Glucose | 5-15 |
| Sodium Acetate | 1-5 |
| Corn steep liquor | 1-8 |
| Yeast extract | 0.05-4 |
| NaCl | 10-100 |
| Cysteine | 0.05-0.15 |

TABLE 5
Medium Containing Sucrose as a Carbon Source
and Containing Nutrients

| Ingredients | Amount (g/L) |
|---|---|
| Sucrose | 5-15 |
| Sodium Acetate | 1-5 |
| Corn steep liquor | 1-8 |
| Yeast extract | 0.2-2 |
| NaCl | 10-100 |
| Cysteine | 0.05-0.15 |

TABLE 6
PEORMC Nutrient Medium

| Ingredients | Amount (g/L) |
|---|---|
| NaCl | 5-100 |
| MgCl₂ | 0.2-0.4 |
| KH₂PO₄ | 0.2-0.4 |
| KNO₃ | 0.2-0.4 |
| NH₄Cl | 0.2-0.4 |
| Resazurin | 0.002-0.003 |
| Cysteine HCl | 0.2-0.4 |
| Sodium acetate | 0.5-1.5 |
| Corn steep liquor | 1-10 |
| Casein peptone | 0.5-1.5 |
| Malt extract | 0.5-1.5 |

Various enrichment media containing specific nutrients are also used to selectively enrich the microbial consortia. For instance, different media are prepared with and without a carbon source (e.g., glucose, fructose, sucrose or maltodextrin), salt (sodium chloride), yeast extract, malt extract, casein peptone, sodium acetate, sodium lactate, corn steep liquor (as the primary source for lactic acid, vitamins and trace minerals), and minerals nutrients (e.g., nitrates, phosphates, ammonium chloride), plus any other minerals, trace minerals and vitamins. The ingredients are dissolved in water and the pH of the media is adjusted to about 7.0 to 8.0. The alkali used to adjust the pH of the media may be sodium hydroxide, potassium hydroxide or ammonium hydroxide. These media are used for screening the microbes and for enhancing the growth of anaerobic bacteria. In some preparations, oil from the formation is supplemented to the media to serve as a carbon source. The requirement of complex nutrients is systematically minimized in successive selection steps. The media for laboratory culturing of the microorganisms are generally prepared by mixing all the desired components, except any reducing agents, in a 1-2 liter glass flask. The flask containing the medium is heated up to 90° C. to drive off as much oxygen a possible using any suitable heating method. For example, a magnetically stirred hot plate or a water bath with a temperature regulator set at the desired temperature may be used. The medium is purged with anaerobic gas comprising of an inert gas such as N₂. The medium is then dispensed into anaerobic vials, using a sterile 10-50 ml disposable pipette, for example. The vials are then securely plugged and sealed (e.g., with rubber stopper and aluminum crimps). The vials and media are then sterilized. For example, they may be autoclaved at 121±5° C. and 15-20 psi pressure for 20-45 minutes. After cooling the vials, an appropriate amount of the reducing agent (e.g., cysteine hydrogen chloride) is then added under sterile conditions. In scale-up, a larger glass reactor (e.g., 5-10 liters capacity) is used.

Development of Stable Bacterial Consortium From Enrichment Media. While continuing to keep the sample in an anaerobic environment an enrichment process is started at approximately the same temperature as the formation temperature of the oil well to be treated. Equal aliquots of the formation water sample are transferred to each of about 4-12 anaerobic media vials (e.g., 4-6 mL each) containing, variously, nutrient rich screening media, mineral rich screening media, and other media as exemplified in Tables 2-6 and further described below. Sodium chloride is added in concentrations from 0.5 wt % to 10 wt %. The enrichment of consortia may be carried out in duplicate or triplicate, if desired. The temperature of the enrichment is maintained at the selected reservoir temperature for approximately 2-7 days for incubation to produce one or more vials containing respective consortia.

Of the original screening vials, the ones that contain the surviving populations are used for further screening. As mentioned above, the temperature of incubation is based on the approximate formation temperature of the particular well to be treated. For instance, in some cases the incubation temperature is 80° C., in another case it is 43° C. and in still another case it is 67° C., and so forth.

After temperature screening, the vials containing the consortium of interest are subjected to further selection steps. These microbes are collectively are referred to as the Stage I consortium. For the purposes of this disclosure, the term “consortium” as in “Stage I consortium,” refers to the product of cultivating microorganisms under at least one chosen condition. The microorganisms may be either a single species or strain of bacteria, for instance, or they may be a mixed multi-strain culture. Typically, the Stage I consortium is a subset of the original microorganisms present in the formation water sample. As desired, the best performing microbes of the Stage I consortium may be further selected for desirable characteristics by selecting the right mix of nutrients for inclusion in a combined mineral rich and nutrient rich media. For instance, to suppress the population of sulfate-reducing bacteria in the Stage I consortium, potassium nitrate is added and sulfate compounds are avoided. Sulfate reducing bacteria can produce H₂S gas that can sour the oil reservoir. To select for a biosurfactant-producing consortium, in subsequent enrichments oil is added. Additional growth stimulators may be used to identify certain media components that can cause the microbes to favor production of a desirable metabolite to the exclusion of another less desirable metabolite. As an example, a nutrient medium may be modified in some instances to initially accelerate acid and carbon dioxide production to the exclusion of biosurfactant production. Then later, the microbes revert to production of biosurfactants rather than acid and carbon dioxide, due to a change in the available carbon source. After all of the desired selection steps have been carried out, the resulting consortium of interest is called the Stage II consortium.

The microbes in the Stage II consortium are further stabilized in mineral media with minimal addition of nutrient rich components such as casein peptone, yeast extract, malt extract and corn steep liquor. Selection of the microbial consortium for which the requirement for complex nutrients can be reduced is accomplished by systematically minimizing several complex nutrients in the media in successive selection steps. An example of a low-nutrient mineral rich formulation is given in Table 3. This process selects for microbes that can potentially survive downhole without an abundance of rich nutrients. The best performing microbes of this consortium (i.e., the greatest population as measured by optical density of the culture) is called the “Stage III consortium.” The microbial consortium that exhibits the best performance in low-nutrient mineral rich media are then subjected to culturing in various media formulations that are nutrient rich, to obtain a microbial consortium that grows rapidly, as further described below.

In some embodiments, a selected stable consortium contains strains that possess basophilic, thermophilic and anaerobic properties. The microbial strains in the Stage III consortium are examined and characterized as part of the PEORMC, in some applications. For example, the bacterial strain or strains in the selected stable consortium may be identified using standard 16S rDNA sequencing techniques. Using other standard laboratory techniques, materials and equipment, the media in which the microbes are grown are tested for metabolites. Some of these studies are conducted using formation water and oil as components of the media, and are conducted in such a way as to represent actual field conditions. The selected cultures are evaluated in various combinations in formation water and oil containing media. These tests include qualitatively and/or quantitatively determining the volatile fatty acids, solvent, biosurfactant, biopolymer, and gases that are produced by the microbes. In some instances the various microbes are isolated and the specific metabolites produced by each strain are determined. The consortium that produces the most desirable metabolites is selected for the scale-up process. Desirable metabolites are those which act on the formation oil to reduce viscosity and/or to produce useful quantities of gas. Without being limited to a particular theory to explain the beneficial properties of the selected microbes, it is believed that the volatile fatty acids reduce the viscosity of the oil in the selected oil well, that carbon dioxide and hydrogen gases produced by the microbes help in sweeping the oil from the formation. It is also thought that the biosurfactant reduces surface tension, thereby mobilizing the oil, and that the biopolymer increases the viscosity of the water, which helps increase the displacement of the oil.

Mass Scale Production of Microbes. The mass culturing of the selected stable consortium is done in an anaerobic bioreactor with high temperature and pressure control facilities. The capacity of the bioreactor may be 1200 liters, for example. The bioreactor is sterilized (e.g., by autoclaving) and then the sterile medium is aseptically introduced into the bioreactor. The nutrient mixture that is used contains a combination of the best mineral rich media components and the best nutrient rich media components, as determined in the preceding screening and selecting steps in some embodiments. One example of a suitable nutrient and mineral rich formulation is given in Table 3. The medium is sterilized by membrane filtration or autoclaving. For example, the medium may be autoclaved at 121±5° C. and 15-20 psi for 20-45 minutes. The sterile medium is then introduced into the pre-sterilized bioreactor or fermentation tank. The bioreactor containing the sterile media is sparged with sterile inert gas, e.g., nitrogen. The impeller speed, gas purging, pH regulation and temperature controls are appropriately set. For example, they may be set at 10-40 rpm, gas flow rate of 0.1-0.5 vvm, pH 7.0-8.0, and about 40° C. to 80° C., respectively.

Prior to culturing the selected microbial consortium in the large capacity bioreactor or fermentation tank, the microbe population is expanded in one or more intermediate transfers. For instance, a 100-200 ml consortium may be transferred to a 2-5 liter bioreactor and grown to active log phase, and the 2-5 liter culture then transferred to a 100 liter bioreactor. After culturing the consortium in the 100 liter bioreactor, it is then transferred anaerobically to the 1200 liter capacity bioreactor. At all times during the intermediate culturing the consortium is maintained at the temperature of the formation water from which the microbes originated or were derived. All transfers are carried out anaerobically to prevent the introduction of oxygen into the bioreactors. For the purposes of this disclosure, microbes “derived” from a sample of formation water include the progeny of microbes that were originally obtained directly from such sample.

In the bioreactor, the consortium is maintained at the formation water temperature until the cell population has reached an optimal level, for example, in some cases the consortium will reach about 10⁸ to 10⁹ cells per milliliter or an OD of approximately 0.5-2. This will typically take from about 16 to 72 hours (depending on the type of consortium) in the 1200 liter bioreactor. As in the preceding steps, the consortium and media are kept at all times in an oxygen-free environment, by sparging the incubation bioreactors with nitrogen and including reducing agents in the media, for example, cysteine HCl. Nitrogen is sparged into the bioreactors until such time as the consortium starts producing its own gasses (e.g., CO₂ and CH₄). The resulting product is termed the Stage IV consortium or the inoculum, and is suitable for mixing with a large volume of bulk medium for carrying out the PEORMC at a selected oil well site. The Stage IV consortium may contain a single bacterial species or strain, or in some instances it may comprise a consortium of mixed multi-strain microorganisms, depending on the particular oil reservoir from which they were derives and depending on the specific screening and selection criteria that are employed to yield the selected microbes.

Developing the PEORMC Medium. The medium used in the bioreactor for preparing the Stage IV consortium (inoculum) is modified for supporting growth of the selected microbes in the downhole environment by elimination of costly carbon sources, minerals and trace minerals, and addition of commercially available low cost ingredients such as yeast extract (Sensient), casein peptone (Food Ingredient Inc.) and corn steep liquor (Cargill). The formulation of an exemplary PEORMC bulk medium is given in Table 6. The pH of the medium is adjusted to 7-8.

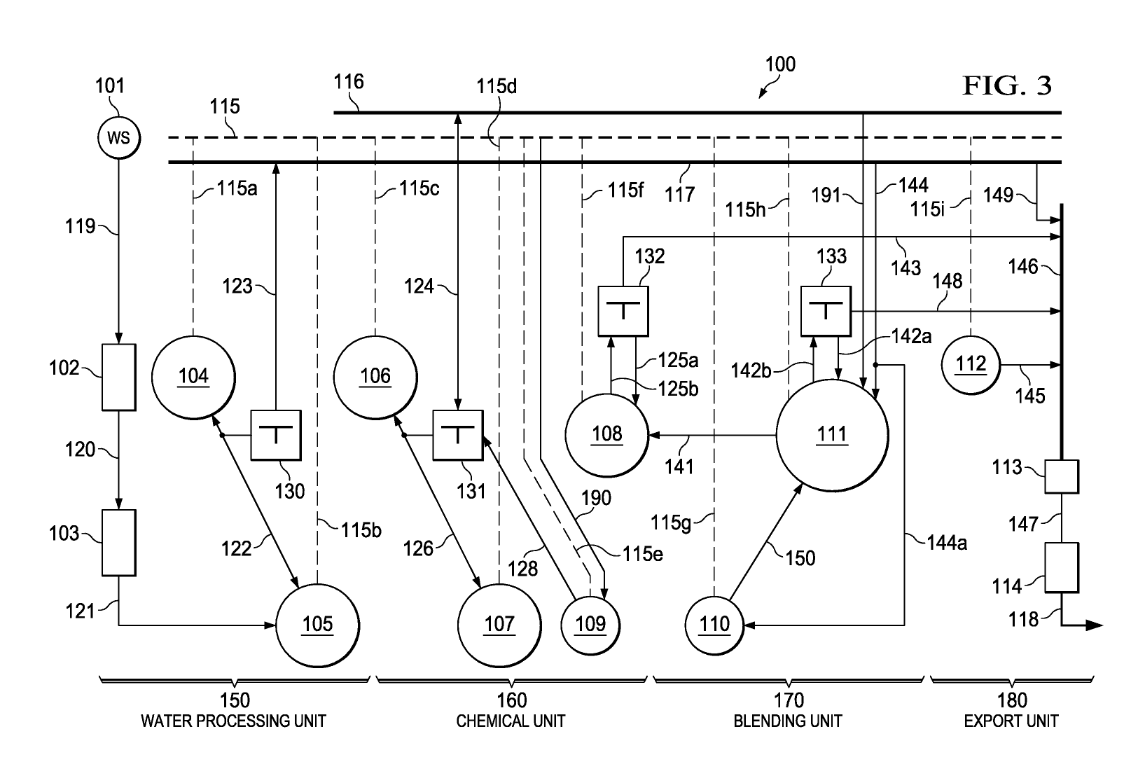

A large volume (e.g., about 40,000 to about 150,000 liters) of the PEORMC bulk medium is prepared for delivering to the oil well site for combination on-site with the microbial inoculum (Stage IV consortium). The ingredients are mixed in well water. The dissolved oxygen of the well water is naturally very low (generally less than 0.5 ppm). The complex media components, including CSL, yeast extract, malt extract and casein peptone are prepared in a manner that maintains strict anaerobic conditions. For example, in some embodiments these complex media components are prepared separately and blended in a blending assembly as shown schematically in FIG. 3, which maintains anaerobic conditions during production of the medium. Sugar and mineral salts are then blended into the complex media mixture. Care is taken to ensure that air is not introduced into the medium and that there is no microbial contamination of the medium during preparation or transfer into the large-capacity delivery tanks. Finally a reducing agent is added to the medium.

With reference to the simplified schematic flow diagram in FIG. 3, a representative medium blending system 100 is configured for attaching to a supply of fresh well water 101 and for connecting to one or more ISO tankers for receiving the completed medium via discharge line 118. The system generally comprises a water processing unit 150, chemical unit 160, blending unit 170 and export unit 180.

Embodiments of water processing unit 150 include, in series flow arrangement, water source well 101, discharge line 119, ultra-violet light sterilization system 102, irradiated water line 120, 100 micron filter 103, sterile feed water line 121, first water holding tank 105, transfer line 122, second water holding tank 104, pump 130 and discharge line 123. Tanks 104, 105 may be 8000 gallon plastic tanks, for example.

Embodiments of chemical unit 160 include first holding tank 106 connected via transfer line 126 to second holding tank 107, gear pump 131, discharge line 124, bulk chemical tank 109, transfer line 128, feed water line 190, nutrient concentrate tank 108, pump 132, suction line 125a, recirculating line 125b, and discharge line 143 that is connected to manifold 146, for discharging the contents of nutrient concentrate tank 108. Tanks 106, 107 may be 8000 gallon plastic tanks, for example.

Embodiments of blending unit 170 include secondary chemical tank 110 and blending tank 111 connected by transfer line 150. Blending unit 170 also includes feed water lines 144, 144a, chemical feed line 191, pump 133, recirculating lines 142a and 142b, chemical transfer line 141, loadout discharge line 148 for discharging the contents of tank 111 into manifold 146. Between tank 111 and tank 108 is transfer line 141, for transferring the blended contents of tank 111 to tank 108. Tank 110 may be a 250 gallon stainless steel tank, and blending tank 111 may be a 5000 gallon stainless steel tank, for example.

Embodiments of export unit 180 include dosing tank 112, discharge line 145 that connects to manifold line 146, flow meter 113, connection line 147, microfiltration unit 114, and export line 118 for loading ISO tankers with the anaerobic blended medium. Dosing tank 112 may be a 250 gallon stainless steel tank, for example. The microfiltration unit 114 contains a five-micron membrane filter, for example.

The system 100 further comprises a nitrogen manifold 115 and distribution lines 115a-i, which are coupled to water tanks 104, 105, holding tanks 106, 107, bulk chemical tank 109, nutrient concentrate tank 108, secondary chemical tank 110, blending tank 111 and dosing tank 112, respectively.

The blending system 100 also comprises a manifold 117 and a manifold 116. Manifold 117 is in fluid flow communication with water lines 123, 144, 149 and 190. Manifold 116 is in fluid flow communication with chemical lines 124 and 191.

When system 100 is used to prepare bulk medium for the PEORMC, the flow of nitrogen into manifold 115 and distribution system 115a-i is begun to purge air that is initially in the blending system. After purging, tanks 104, 105, 106, 107, 108, 109, 110, 111 and 112 are blanketed with nitrogen at 1 psig. The nutrient medium blending process then proceeds by drawing water from the water supply well 101 via line 119 and treating it by exposure to UV radiation as it passes through the light source filter 102. The water is then filtered through filtration unit 103 (containing a 100 micron filter, for example), and the filtered water is then transferred into the water holding tanks 104 and 105. The desired amount of bulk chemicals (e.g., sugar, sodium chloride, corn steep liquor) are loaded into tank 109 (e.g., through a manway) and tank 109 is then purged with nitrogen. Water is blended into tank 109 via water line 190, and the resulting mixture is then pumped into holding tanks 106 and 107 by pump 131. Other chemicals (e.g., buffer salts, minerals, casein peptone, and malt extract) are added (e.g., through a manway) into the secondary chemical tank 110, and then purged with nitrogen. Water is then blended into tank 110 via water lines 144, 144a, and the resulting mixture is pumped into the blending tank 111. The desired amount of water from holding tanks 104 and 105 is transferred through manifold 117 and line 144 to the blending tank 111. The desired amount of liquid chemical solution in holding tanks 107 and 108 is then transferred via line 124 to manifold 116 and line 191 into blending tank 111. Blending is accomplished in tank 111 by circulating the fluid through pump 133 via suction line 142a and discharge line 142b. After blending in tank 111 for a predetermined time, the contents are discharged into the nutrient concentrate holding tank 108.

When loading and transport of the nutrient medium is required, the desired amount of water is loaded to the receiving tank (e.g., an ISO tanker) from water holding tanks 104 and 105 through line 123, manifold 117, line 149, export manifold 146, through meter 113, filter 114 and export line 118 to the receiving tank (not shown). For example 9-10 parts of water are combined with 1 part of concentrated nutrient mix. The desired volume of (e.g., a 10× concentrate) nutrient concentrate in tank 108 is then discharged through suction line 125a and pump 132 to line 143, to manifold 146, and thence through meter 113, filter 114, an export line 118 to the ISO tanker. Reducing agents and pH balancing chemicals are added to the flowing nutrient concentrate, as needed, from dosing tank 112 via line 145, to ensure that it meets desired specifications (e.g., pH of 7.0-8.0 and an oxidation redox potential of −50). This mixing methodology enables pre-blending of the larger quantities of bulk chemicals, for example sugar, salt and corn step liquor, which in turn reduces the loading time for each successive ISO tanker. Alternatively, the nutrient media may be prepared by any other suitable method to provide the necessary quantity of aseptic medium for treating an oil reservoir.

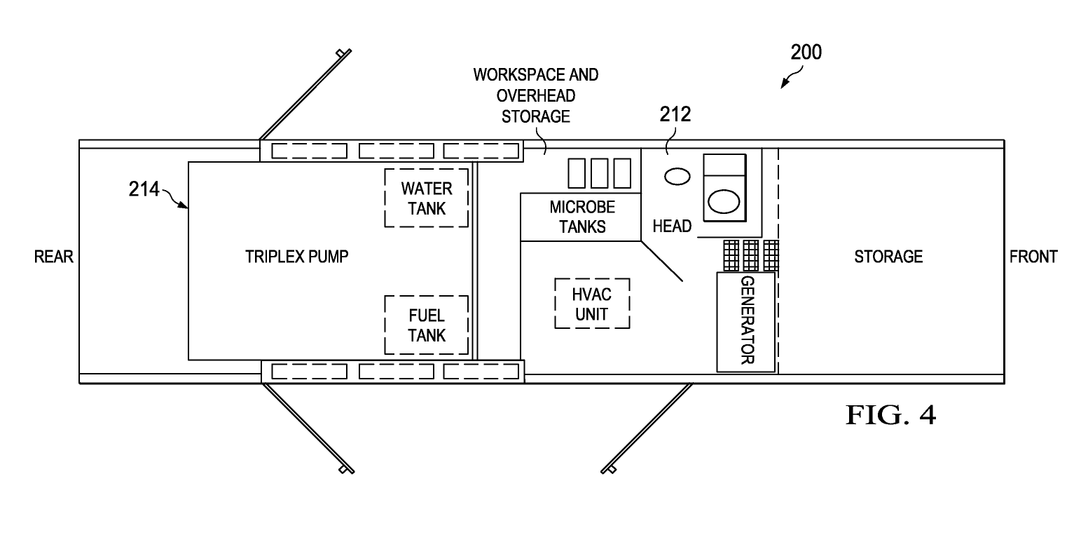

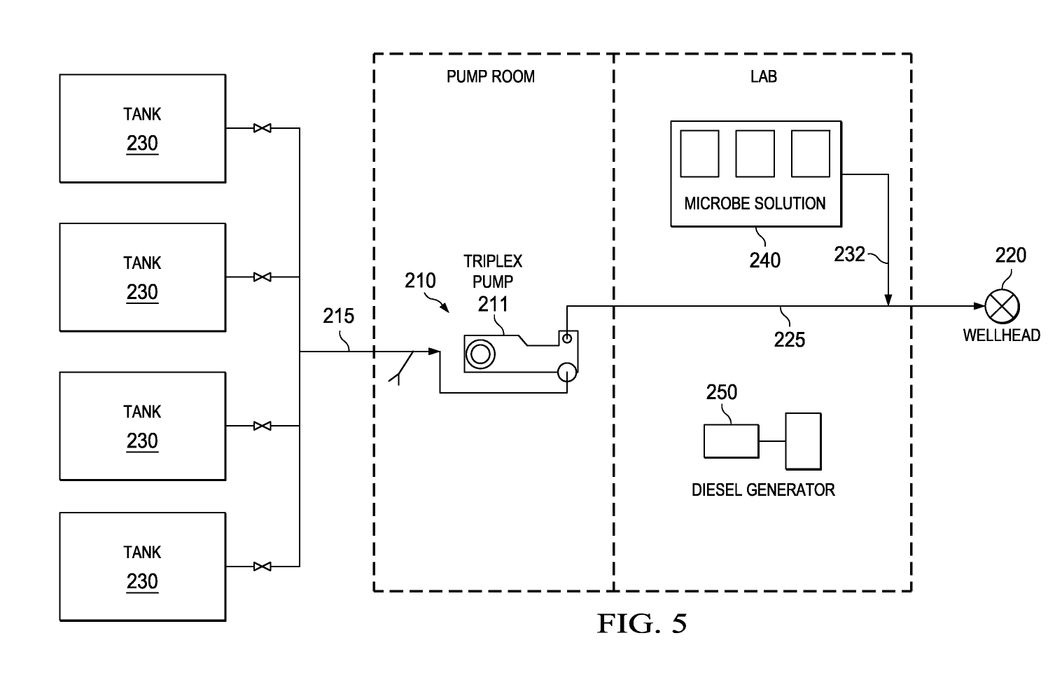

Transportation of Microbial Consortium and Nutrient Media. The fully grown mesophilic and thermophilic anaerobic consortium are transferred aseptically from the bioreactor to specially designed nitrogen blanketed containers in a mobile field laboratory assembly 200, which is schematically illustrated in FIG. 4. For example, three 60 liter tanks of the selected consortium, along with their supporting equipment are transported to the treatment site in the field laboratory unit 212. The field laboratory assembly 200 also contains a triplex pump unit 214 for pumping the PEORMC bulk medium and the consortium into the oil wellbore and into the formation. The PEORMC bulk medium is transported at ambient temperature to the well site in 24,000 liter ISO tanks that are nitrogen blanked. The ISO tankers and the mobile field laboratory assembly are aligned at the well site as is shown by FIG. 5.

Pumping of Bioslurry and Injectivity Test in Selected Wells. Prior to pumping the consortium and medium mixture (“bioslurry”) into the formation, an injectivity test at the desired pressure (e.g. not more than 1600 psig) using a pre-mixed oxygen-free salt water (with the same salinity as the formation) is conducted at the wellsite to ensure that the formation is capable of accepting the bioslurry.

One suitable way to anaerobically combine and pump the consortium and the bulk medium is by using a pumping assembly 210 as shown in the schematic process flow diagram of FIG. 5. Positioned near the selected oil well, the pump 211 draws the bulk medium from one or more tanks 230 through line 215, and pumps the medium to wellhead 220 through line 225. One or more ISO tanker trucks, each holding about 24,000 liters of PEORMC medium in a stainless steel tank 230 may be transported to the well site for this purpose. The microbial consortium is fed from one or more consortium tank 240 into line 225 and mixes with the flowing PEORMC medium. The consortium may be fed into line 225 by a venturi valve 232 or by a positive displacement dosing pump, or any other suitable means that avoids introduction of air or contaminating microbes into the stream. In some embodiments, the pump 211 is a triplex pump capable of operating at about 1600 psi and about 42-70 gallons per minute. In some applications, about 100-300 liters of consortium is injected into about 750 bbls of PEORMC medium at an injection rate of about 1-1.5 bbls/min. Quality control of the microbial consortium, PEORMC nutrient medium and the bioslurry may be accomplished through a set of instrumentation probes that are inserted into the low pressure triplex pump suction line and high pressure discharge line. The corresponding instruments continually measure such parameters as oxidation redox potential (ORP), temperature, pH, conductivity, pressure, and flow rate. The data collected from these probes may be continually displayed on analog meters located inside the mobile field laboratory and recorded digitally to a notebook computer for viewing, storage and for display on the Internet, as desired. Alternatively, any other suitable type of pump may be employed in the process.

In some embodiments, a specific sequence is followed for injection of nutrient medium and the selected consortium (inoculum) into the selected well to ensure that no contaminants are introduced and that the medium and inoculum remain anaerobic during the entire process. The total volume of the PEORMC nutrient medium plus inoculum to be injected into the oil zone of the well depends on pay zone thickness, porosity, radius of treatment and injectivity. As a pre-flush, water is introduced into the well in an amount corresponding to about 10 vol % of the total nutrient medium volume. This is followed by pumping nutrient medium in the amount of about 20 vol % (of the total volume of medium) into the well. Then the inoculum (in the amount of about 0.2-1.0 anaerobically vol % (of the total treatment volume) is mixed into the next approximately 60 vol % of the medium as it continues to flow through line 225. The bioslurry constitutes approximately 60% of the total medium volume (also referred to as the treatment volume). After all of the inoculum is injected into the flowing medium, the flow of nutrient medium continues until the remaining approximately 20 vol % of the medium has been pumped into the well. Finally, a volume of water is injected sufficient to displace into the reservoir the preceding portion of nutrient medium that remains in the tubing/casing. In some cases this final volume of water is supplemented with alkali to counteract any potentially corrosive residual media components, and to minimize microbial growth in the wellbore. The water, medium (only), and bioslurry are all injected into the well at a rate of from about 0.5 to about 2.0 barrels per minute. In some cases, the injection pressure does not exceed 1600 psig. For example, in a treatment volume of 400 barrels, a pre-flush of 40 barrels of salt water is followed by 80 barrels of nutrient medium, followed by 240 of bioslurry and remaining 80 barrels of nutrient medium.

Incubation of Culture in the Formation. After injection of the desired treatment volume, the well is shut in, and the selected consortium is allowed to incubate in situ for about one to about three weeks. In some applications, the presence of salty formation water (i.e., up to about 10% sodium chloride) is compatible with the PEORMC medium, and provides a favorable environment for the selected PEORMC consortium to proliferate and produce beneficial metabolic products. By action of the selected microbes, supported by the PEORMC nutrient medium, the viscosity of the oil in the formation is reduced due to the miscibility of CO₂ in oil under certain pressure and temperature conditions. Interfacial tension will be lowered by the production of biosurfactant and the sweep efficiency will be improved due to the production of biopolymers and gases such as CO₂, CH₄ and H₂. The metabolite mix will cause change in wetabilities which in turn will allow more oil and less water to be produced. The production of fatty acids leads to the clean up and removal of potential formation skin damage thereby increasing the total fluid production.

The action of the microbes also potentially improves the quality of the recovered oil. After completion of the incubation period, the well is then opened for production and parameters such as the fluid production rate, water cut, microbial population in produced water, conductivity, acids, pH, oil viscosity and specific gravity, may be monitored. In some instances the oil production rate is enhanced by the PEORMC up to two to four fold, or more.

Use of the PEORMC is generally applicable to wells that fit a certain profile, based on their reservoir and fluid properties as described above with respect to the general screening criteria. It should be understood that in many cases other oil wells may also be successfully treated with the same microbial consortium if the wells share the same or similar reservoir and fluid properties with the well from which the microbes were derived.

### EXAMPLES

### Example 1

### Screening and Testing of 43° C. Formation Sample

In one test, an anaerobic microbial consortium was screened and enriched from a sample of formation water taken as described above from an oil well in which the temperature of the formation was about 43° C. The formation water contained about 0.5 wt/v % salt (NaCl). The screening and selection procedure was performed as generally described above, except in this case the temperature of incubation was kept at 43° C., which corresponded to the formation temperature. The consortium selected from the 43° C. well was additionally tested at several growth temperatures ranging from 37° C. to 65° C. and the resulting metabolic products were analyzed. Significantly, the consortium obtained from the 43° C. formation showed higher production of gases including hydrogen and carbon dioxide as per volume of media used. The volume of gas production varied in the range of three to four times the liquid volume. Another significant feature of the 43° C. developed consortium was the utilization of acetate and conversion to butyrate in nutrient rich medium, the components of which are shown in Table 2. The measurement of metabolites was performed by using gas chromatography (HP Model 6890 gas chromatograph) using a flame ionization detector (FID) and a capillary column.

### Example 2

### Screening and Testing of 67° C. Formation Sample

Another anaerobic microbial consortium was taken from a sample of formation water in another oil well, and was screened and enriched, as described above. In this instance the sample was taken from an oil well in which the temperature of the formation was about 67° C. The formation water contained about 6-8 wt % salt (NaCl). The consortium in various screening media were incubated at 67° C. for selection and enrichment of the microbes.

### Example 3

### Controlling the Surface Tension Activity of Selected Microbes

Enriched consortium were evaluated for surface tension activity in medium formulated as described in Table 3. The reduction in surface tension of the culture broth was significant (from 49 to 37 units) when oil was used as a sole carbon source. Although the amount of acid production was reduced when oil was used as a carbon source, these findings suggest that when sucrose is added to the bulk treatment medium at the well site, a higher amount of acids and gases are produced and later the oil in the formation may potentially contribute to biosurfactant production downhole. Appreciable use of oil by the microbes as a carbon source will then favor production of biosurfactants in the formation. Thus, this selected culture demonstrated that it produces three key metabolites that contribute to the enhanced oil recovery. The isolated consortium showed good growth over a one day period in both nutrient rich media and media containing lower-cost nutrients, such as the medium of Table 6. The selected consortium was identified as Garciella petrolaria by using standard 16S rDNA sequencing techniques.

### Example 4

### Enhancement of Oil Recovery in a High Salinity Reservoir

A bulk nutrient medium is made by combining the components listed in Table 6 with RO (reverse osmosis) water to 1000 mL, adjusting the pH to 7.0-7.5. The percentages are by weight-volume. The nutrient mix is autoclaved at a temperature of 121° C. and 15-20 psi pressure for 20-25 minutes. The nutrient mix is dispensed hot in anaerobic vials while purging with nitrogen. The containers are sealed and autoclaved. After autoclaving, the reducing agent (cysteine HCl) is added in the nutrient mix that is kept at the reservoir temperature in a temperature controlled shaker or oven. The medium is inoculated with a consortium containing one or more bacterial species (e.g., Clostridium bifermentans and/or Garciella petrolaria), to prepare the inoculum for scale up or mass production for carrying out the PEORMC for a selected oil well. The microbes are obtained from the oil well formation water, in accordance with the above-described general procedures.

The target oil well selected for receiving the PEORMC meets all of the reservoir and fluid properties criteria set forth above in Table 1. The inoculum containing the selected thermophilic/mesophilic anaerobic consortium is transferred aseptically from the large (1200 liter) bioreactor to nitrogen blanketed containers with continuous purging of nitrogen. The containers are sealed and transported to the well site. The total volume of inoculum medium to be injected is determined as described above, and the same strict anaerobic injection protocol as described above is employed for treating the well.

Without further elaboration, it is believed that one skilled in the art can, using the description herein, utilize the invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A process for enhancing recovery of oil from a subterranean reservoir, comprising:
(a) selecting an oil well reservoir having a treatment zone and meeting the following criteria:

| | |
|---|---|
| Type of formation: | sandstone or carbonate |
| | with dense natural fracturing |
| Presence of gas cap: | no |
| Stage of exploitation: | mature |
| Depth of occurrence: | <10,000 ft |
| Reservoir temperature: | <90° C. |
| Reservoir pressure: | <300 Kg/cm² |
| Formation water pH: | 4-9 |
| Salinity of formation water: | <12% total salinity or <10% NaCl |

(b) providing an anaerobic inoculum comprising a selected consortium of anaerobic microbes obtained or derived from a reservoir that meets the criteria of (a), wherein the selected microbes have a predetermined growth rate at the reservoir temperature, have a predetermined minimum mineral and nutrient requirements for growth, and produce predetermined amounts and/or kinds of-metabolites;
(c) providing a volume of anaerobic nutrient medium formulated to support the growth of said consortium and meeting at least said predetermined minimum mineral and nutrient requirements, said volume being sufficient to fill said treatment zone of said oil well reservoir;
(d) anaerobically combining said inoculum with said anaerobic nutrient medium to form an anaerobic bioslurry;
(e) displacing said bioslurry into said reservoir treatment zone; and
(f) shutting in the oil well and allowing the consortium to incubate in said zone, to enhance oil recovery from said well.

2. The process of claim 1 wherein, in (d), anaerobically combining said inoculum with said medium comprises anaerobically combining said inoculum with a portion of said anaerobic nutrient medium to form said anaerobic bioslurry while simultaneously pumping said medium into said well.

3. The process of claim 1, wherein (f) comprises shutting in the oil well for about one to about three weeks.

4. The process of claim 1, wherein said consortium comprises one or both of Clostridium bifermentans and Garciella petrolaria.

5. The process of claim 1, wherein, in (a), said formation water has a pH in the range of about 6-8.

6. The process of claim 1, wherein, in (b), said microbes are initially obtained by anaerobically sampling the formation water of said reservoir by anaerobically introducing said sample directly from the reservoir tubing into a sterile container under cover of an inert gas.

7. The process of claim 6, further comprising aseptically injecting a reducing agent into said container under cover of said inert gas.

8. The process of claim 1, wherein step (d) comprises injecting said consortium into said stream through a venturi valve configured to exclude air from said medium.

9. The process of claim 1, wherein said oil well comprises formation water containing up to 12% (w/v) salt.

10. The process of claim 1, wherein, in (a), said reservoir additionally meets at least one of the following criteria:

| | |
|---|---|
| Reservoir rock permeability: | >25 md |
| Crude oil gravity: | >20°API |
| Reservoir oil viscosity: | <20 cp |
| Reservoir rock porosity: | >20%, in the case of sandstone |
| | formation only |
| Water cut: | 30-95% |
| Residual oil saturation: | >25% |
| Source of water: | Natural aquifer or water injection |
| Net pay thickness: | 10-12 meters or less |
| Producing zone: | single |
| Type of lift: | rod pump |
| Swelling clays in formation rock: | none |

12. A system for anaerobically treating an oil reservoir, comprising:
a transportable field laboratory assembly including:
a pumping unit comprising:
a triplex pump having a low pressure suction line configured for coupling to a feed line and a high pressure discharge line configured for coupling to an oil well inlet,
a laboratory unit comprising at least one anaerobic bioreactor having an outlet adapted for coupling to said discharge line,
optionally, a power unit containing a generator for powering the system; and
a housing enclosing at least said laboratory unit and said pumping unit.

13. The system of claim 12, further comprising:
a plurality of instrument probes operationally positioned in said suction and discharge lines for measuring predetermined operational parameters during operation of said system; and
a plurality of instruments in electronic communication with said probes, respectively, for receiving data from said probes and reporting information about the respective parameters.

14. The system of claim 13, wherein said instruments are configured for measuring and reporting at least one parameter selected from the group consisting of oxidation redox potential (ORP), temperature, pH, conductivity, pressure, and flow rate.

15. A system for producing an anaerobic nutrient medium, comprising:
a water processing unit comprising at least one water holding tank, and configured for providing a sterile water stream;
a chemical unit comprising:
a bulk chemical tank configured for receiving bulk chemicals and said sterile water stream,
at least one chemical holding tank configured for receiving a bulk chemical solution from said bulk chemical tank and for providing a chemical stream, and
a nutrient concentrate holding tank configured for receiving a blended stream and for providing a nutrient concentrate stream;
a blending unit comprising a secondary chemical tank, a mixing device and a blending tank configured for receiving said chemical stream and said water stream, and for providing said blended stream;
an export unit comprising a dosing tank and a first manifold adapted for receiving said sterile water stream, said nutrient concentrate stream, and said blended stream, and for receiving a dosing stream from said dosing tank, said export unit also adapted for discharging a nutrient medium;
a second manifold in fluid communication with said water processing unit, said chemical unit, said blending unit, and said first manifold, and configured for receiving said sterile water stream;
a third manifold in fluid communication with said chemical unit and said blending unit, and configured for receiving a chemical solution from said chemical unit; and
a gas distribution assembly in fluid communication with said water tanks, chemical holding tanks, bulk chemical tank, nutrient concentrate tank, secondary chemicals tank, blending tank and dosing tank.

16. The system of claim 15, wherein said at least one chemical holding tank is in fluid communication with said third manifold.

17. The system of claim 15, wherein said secondary chemical tank is configured for receiving said water stream and said blending tank is configured for receiving a secondary chemical stream from said secondary chemical tank.

18. The system of claim 15, wherein said export unit further comprises a digital flow totalizer and a filtration membrane in fluid communication with said first manifold.

19. A system for producing an anaerobic nutrient medium, comprising:
means for providing anaerobic sterile water;
means for providing a bulk chemical solution;
means for providing a secondary chemical solution;
means for blending said water, bulk chemical solution, and secondary chemical solution, to provide a nutrient concentrate;
means for transferring said water, bulk chemical solution, and secondary chemical solution into said blending means;
means for holding said nutrient concentrate;
means for exporting said nutrient concentrate;
means for adjusting the concentration of said nutrient concentrate during exporting of said concentrate; and
means for maintaining anaerobic conditions in said system.

20. A method of producing a nutrient medium, comprising:
(a) providing the system of claim 15;
(b) in said chemical unit, anaerobically preparing a bulk chemical solution comprising NaCl, MgCl₂, KH₂PO₄, KNO₃, NH₄Cl, and corn steep liquor;
(c) in said blending unit, anaerobically preparing a second chemical solution comprising casein peptone, malt extract, sodium acetate, a redox indicator and a reducing agent;
(d) in said blending unit, anaerobically combining predetermined amounts of said first and second chemical solutions and water, to produce a nutrient concentrate;
(e) anaerobically transferring said nutrient concentrate to the nutrient concentrate holding tank of said chemical unit;
(f) in said exporting unit, anaerobically exporting predetermined amounts of said sterile water and said nutrient concentrate into a receiving tank, to yield an anaerobic nutrient medium;
(g) optionally, adjusting the pH and redox potential of said nutrient concentrate during said exporting.

21. The method of claim 20, wherein the anaerobic nutrient medium in said receiving tank comprises the following ingredients and amounts in a volume of about 40,000 to 150,000 liters:

| Ingredients | Amount (g/L) |
|---|---|
| NaCl | 5-100 |
| MgCl₂ | 0.2-0.4 |
| KH₂PO₄ | 0.2-0.4 |
| KNO₃ | 0.2-0.4 |
| NH₄Cl | 0.2-0.4 |
| Resazurin | 0.002-0.003 |
| Cysteine HCL | 0.2-0.4 |
| Sodium acetate | 0.5-1.5 |
| Corn steep liquor | 1-10 |
| Casein peptone | 0.5-1.5 |
| Malt extract | 0.5-1.5 |

22. An anaerobic sampling device comprising:
a sterile sample bottle including a pierceable septum;
a sample bottle container including:
a first injection needle coupled to a bleed line including a valve, and
a second injection needle coupled to a sample inlet line, wherein said container is adapted for receiving said bottle and said first and second needles are adapted for piercing said septum;
a formation water line comprising a valve adapted for coupling to an oil well tubing flow line;
an inert gas inlet line including a valve adapted for coupling to a source of inert gas;
a three-way valve operatively connected to said formation water line, said sample inlet line, and to a fluid bypass line.
